(19) United States
(12) Patent Application Publication
(10) Pub. No.: US 2020/0093659 A1
(43) Pub. Date: Mar. 26, 2020

(54) TETHERED SURGICAL SPONGES

(71) Applicant: Marlene A. Griggs-Warner, Clementon, NJ (US)

(72) Inventor: Marlene A. Griggs-Warner, Clementon, NJ (US)

(21) Appl. No.: 16/211,392

(22) Filed: Dec. 6, 2018

Related U.S. Application Data
(60) Provisional application No. 62/733,699, filed on Sep. 20, 2018.

(51) Int. Cl. A61F 15/00; A61F 13/36

(52) U.S. Cl. CPC A61F 15/001; A61F 13/36

(57) ABSTRACT

The present invention includes the use of a plurality of surgical sponges which are tethered to and contained within individual transparent compartments. The compartments are connected together, side-by-side, in series, to form a single unit line of compartments in which the surgical sponges are located. A pressure fit closure is provided to seal the sponges within their respective compartments.

### RELATED APPLICATION

This application claims the benefit of application Ser. No. 62/733,699, filed on Sep. 20, 2018.

### FIELD OF THE INVENTION

The present invention relates to surgical operations and, specifically, to a system for utilizing and accounting for surgical sponges during such operations, in order to assure that all sponges are removed prior to surgical closure, thus avoiding a sponge being left in the patient

### BACKGROUND OF THE INVENTION

The use of surgical sponges or equivalent absorbent pads during invasive surgical operations is a necessary part of most such procedures. These sponges differ in size and dimensions, e.g. large and small lap sponges 18″×18″; Raytec gauze sponges 4″×18″; 4″×12″ to smaller neuro sponges, 100% absorbent cotton material. It is imperative that all sponges placed within a patent during a surgical procedure be removed prior to closure of the patient. Prior to closure, a sponge count is vital in order to ensure that one or more sponges have not been left in the patient.

Despite best efforts to account for all sponges used in an operation, there are times when one or more sponges may be inadvertently left in the patient. When this is discovered, it is usually necessary to open up the previously closed incision, locate and then remove the unaccounted for sponge or sponges.

There have been prior, different attempts to account for surgical sponges used during surgical operations, for example by utilizing sponges connected to and stored in a single dispenser, U.S. Pat. No. 4,903,837; by stringing a plurality of sponges together, U.S. Pat. No. 9,089,366; by connecting sponges to a common junction member, U.S. Pat. No. 9,808,315; and by accounting for sponges removed from a dispensing strip by alphanumeric identifiers, U.S. Pat. No. 9,872,732. However, these prior and similar systems are either unreliable in that untethered sponges are often difficult to retrieve, are difficult to position during operations, or are cumbersome to place or use, especially during delicate surgical procedures.

To date, there are no adequate, practical, effective and reliable accounting systems for sponges used in surgical operations.

### SUMMARY OF THE INVENTION

It is thus the object of the present invention to provide an accounting system of tethered surgical sponges which overcomes the disadvantages and limitations of prior surgical sponge accounting systems.

This and other objects are accomplished by the present invention, a plurality of surgical sponges which are tethered to and contained within individual transparent compartments. The compartments are connected together, side-by-side in series, to form a single unit line of compartments which comprise the tethered surgical sponge accounting system.

The novel features which are considered as characteristic of the invention are set forth in particular in the appended claims. The invention, itself, however, both as to its design, construction and use, together with additional features and advantages thereof, are best understood upon review of the following detailed description with reference to the accompanying drawings.

### BRIEF DESCRIPTION OF THE DRAWINGS

FIG. 1 shows the tethered surgical sponges of the present invention in position on a surgical drape, prior and ready for use.

FIG. 2 is the tethered surgical sponges of the present invention being utilized during a surgical procedure.

FIG. 3 is the tethered surgical sponges of the present invention illustrating the collection of soiled sponges following the surgical procedure.

### DETAILED DESCRIPTION OF THE INVENTION

The tethered surgical sponge accounting system of the present invention comprises a plurality of separate, independent, transparent compartments 2, 4, 6, 8, and 10 connected to each other, side-by-side in series, to form a single unit line of compartments 1. Unit 1 has top opening 12 and reinforced closed bottom 14. Closure means, in the form of a Ziploc® type, pressure fit closure 17 is provided along top opening 12, in order to close off the compartments. End member 16 is provided with grommet holes 18 and 20 so as to allow unit 1 to be hung up.

Surgical sponges 22, 24, 26, 28, and 30 are located within compartments 2, 4, 6, 8, and 10 and these sponges are connected to their respective compartments by flexible tethers 23, 25, 27, 29, and 31. The tethers can be fabricated of cloth material, strings, or equivalent flexible lines.

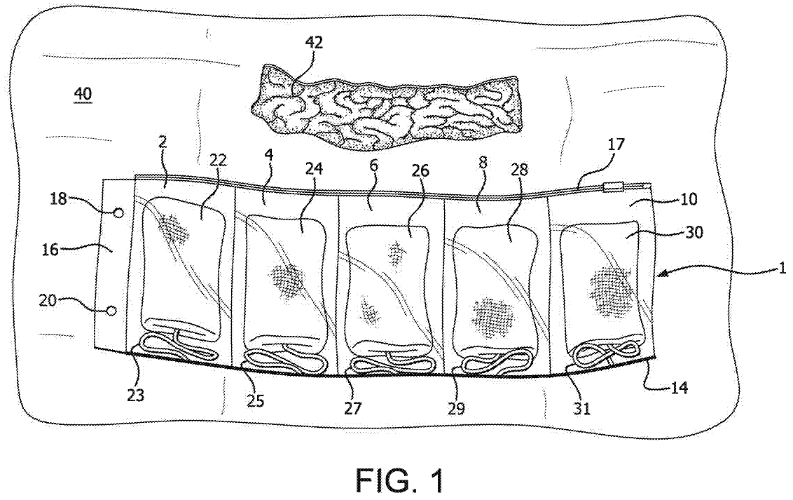

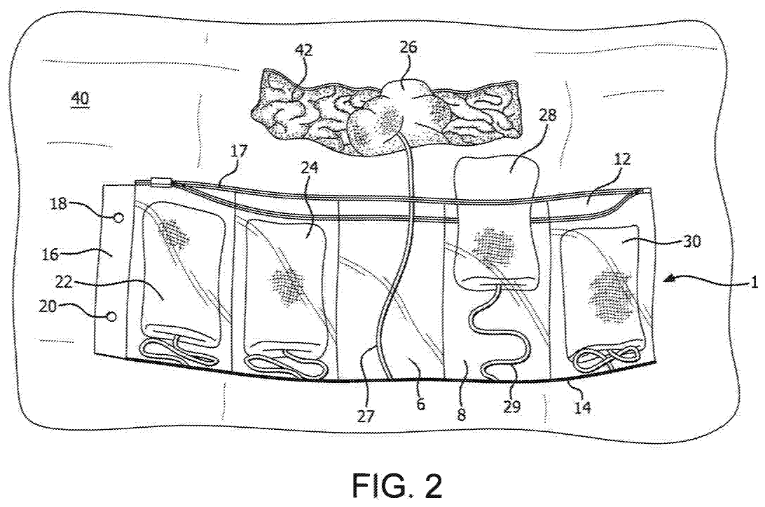

In use, as best seen in FIG. 1, unit 1 is positioned on surgical drape 40. It can be fastened to the drape by Velcro® which may be attached to the back of unit 1 and the drape, or by equivalent attachment means. In this position, surgical sponges 22, 24, 26, 28, and 30 can be easily and efficiently removed from their respective compartments, as needed during the surgical procedure, and placed within the patent's open wound 42. Each sponge remains attached at all times to its respective compartment by its respective tether. FIG. 2 illustrates sponge 26 positioned in wound 42, the sponge remaining attached to compartment 6 by tether 27. Sponge 28, connected to compartment 8 by tether 29, is shown as being the next sponge to be used in the procedure.

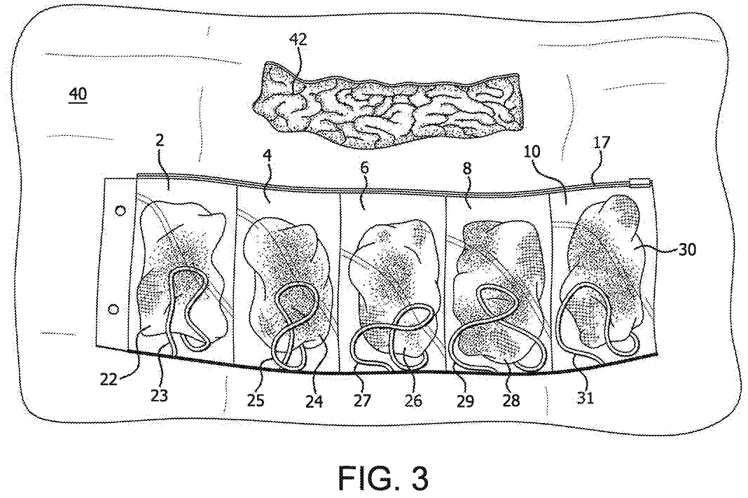

After sponges are saturated or blood soaked, they are returned to their respective compartments, as seen in FIG. 3. All sponges are accounted for when each is positioned in their respective compartments. Pressure fit closure 17 is then secured for sanitary reasons, to ensure that the compartments containing the sponges are sealed off.

Use of tethered surgical sponges in accordance with the present invention provides a ready and reliable means of accounting for surgical sponges following a surgical procedure. It eliminates gossypiboma/texiloma (sponges left in patients after skin closures) and can be adapted and utilized for any type or variety of surgical sponge.

Certain novel features and components of this invention are disclosed in detail in order to make the invention clear in at least one form thereof. However, it is to be dearly understood that the invention as disclosed is not necessarily limited to the exact form and details as disclosed, since it is apparent that various modifications and changes may be made without departing from the spirit of the invention.

1. A system of tethered surgical sponges comprising:
a plurality of independent, transparent compartments connected to each other, side-by-side in series to each other to form a single unit line of connected compartments, said single unit line of connected compartments having a top opening, a reinforced closed bottom, and closure means comprising a pressure fit attachment for sealing the top opening; the top opening, the reinforced closed bottom, and the closure means all extending the length of the single unit line of connected compartments;
a surgical sponge located in each of said plurality of compartments; and
a flexible tether attached between each surgical sponge and the compartment in which that surgical sponge is located, wherein the tethers at all times remain attached between the surgical sponges and the compartments in which the surgical sponges are located, including upon removal of the surgical sponges from their respective compartments and upon replacement of the surgical sponges into their respective compartments.

2. (canceled)

3. The system of tethered surgical sponges as in claim 1 further comprising grommet openings located at one end of the single unit line of compartments.
